(19) United States
(12) Patent Application Publication
(10) Pub. No.: US 2019/0076632 A1
(43) Pub. Date: Mar. 14, 2019

(54) SUTURABLE WOVEN IMPLANTS FROM ELECTROSPUN YARNS FOR SUSTAINED DRUG RELEASE IN BODY CAVITIES

(71) Applicant: Amrita Vishwa Vidyapeetham, Kochi (IN)

(72) Inventors: DEEPTHY MENON, Kochi (IN); SMRITHI PADMAKUMAR, Kochi (IN); SHANTIKUMAR NAIR, Kochi (IN)

(21) Appl. No.: 16/131,063

(22) Filed: Sep. 14, 2018

(30) Foreign Application Priority Data
Sep. 14, 2017 (IN) 201741032588

(51) Int. Cl. A61M 31/00; D01D 5/00; A61F 2/02; A61L 27/54

(52) U.S. Cl. CPC A61M 31/002; D01D 5/0084; A61L 27/56; A61L 27/54; A61F 2/02

(57) ABSTRACT

The invention discloses a saturable drug delivery implant made of a woven fabric material with variable packing density, dimension and weight. The woven fabric material includes electrospun continuous yarn with individual micro- or nano-fiber yarn made from a single polymer. The yarn is loaded with at least one therapeutic agent to provide controlled and sustained release when the device is sutured to a wall of the peritoneal cavity of a subject. The woven fabric is flexible, biodegradable and configured to be sutured to the wall of a body cavity to provide a sustained and controlled release of therapeutic agent in a subject. In some aspects, a peritoneal implant is sutured to the peritoneal wall cavity for continuous and sustained intraperitoneal release of a therapeutic agent.

### CROSS-REFERENCES TO RELATED APPLICATIONS

This application claims priority to Indian Provisional Patent Application No. 201741032588 entitled “WOVEN FABRIC IMPLANTS FOR INTRAPERITONEAL THERAPY” filed on Sep. 14, 2017, the full disclosure of which is incorporated herein by reference.

### FIELD OF THE INVENTION

The present invention relates to drug delivery devices, method of use thereof, and a method of manufacturing thereof and in particular to suturable woven implants for providing controlled and sustained release of a drug in body cavities.

### BACKGROUND

Polymeric biodegradable implants are desirable for various medical applications demanding localized and sustained release of any therapeutic molecule for several days to months. The use of implantable matrices helps to achieve site-specific and targeted effects in comparison to the conventional clinical strategy of systemic intravenous therapy. Body cavities such as peritoneum, brain, bladder, eye, etc are often primary or secondary sites of several diseases or malignancies. The drug depots implanted at these targeted sites help to prevent or treat these diseases which are confined to the particular body cavity.

The US patent publication US20140363484A1 is directed to the development of a fibrous flexible biodegradable drug loaded polymeric wafer system for local delivery of therapeutic agents in combination. Here, combinations of polymers have been used to deliver multiple drugs, enabling sustained release over a period of one month. The US patent publication US20150037375A1 is directed to 3-dimensional drug-eluting materials comprising therapeutic biodegradable polymer(s), with time-release properties that are described to permit the release of one or more therapeutic agents to a subject over extended time periods. WO2016159240A1 relates to a method of producing biodegradable drug loaded fibers as non-woven structures by electrospinning and using them for local sustained drug release in the body. The US patent publication US20100303881A1 provides electrospun fiber compositions comprising one or more polymers and one or more biologically active agents, wherein adjustments to density of the electrospun fiber composition is suggested to increase or decrease the length of time that therapeutic molecules are released from the composition. All these relate to the use of non-woven, biodegradable, fibrous implants developed through electrospinning for achieving sustained releasing drug depots.

The PCT application WO2008033533 is related to a device having a fabric constructed from an oriented multifilament yarn loaded with a therapeutic agent. A composite drug delivery system composed of a fabric wrapped by a drug laden biodegradable matrix coating is disclosed for facilitating drug delivery within a vascular graft. The fabric provides a mechanical reinforcement while the copolymeric matrix coating elutes the entrapped therapeutic agent/bioactive. It is due to the co-polymer coating a predictable rate of polymer degradation and therapeutic agent release is predicted. The U.S. Pat. No. 8,974,814 is directed to a layered polymeric monofilament fiber drug delivery device, where each layer of the device can contain a different polymer, drug, additive, or any combination thereof. A non-woven fabric comprising of thermally stable absorbable fiber population in disclosed in the US patent publication US20170167064A1, to provide a medical barrier for a range of medical applications.

The main attributes of designing a drug eluting depot which can be implanted within any body cavity are the ability to control and sustain the release of the therapeutic agent for several days and the easiness to fix or implant the device to the body cavity. It is imperative to maintain adequate drug concentrations in the desirable target within cavity or site to attain and prolong the therapeutic activity. Slow and continuous release of the drug becomes important to address the issues caused by drug resistance and cell repopulation, especially for tumor therapy applications. The implant has to be fixed to the body cavity wall to prevent the issues like migration to other sites within the cavity, which could eventually cause nonspecific effects, burst release and organ toxicity.

In this regard, the use of electrospun nano/micro yarn-based matrices that can enable suturability and sustained drug release becomes important. U.S. Pat. No. 9,994,975B2 relates to an electrospinning apparatus and method to fabricate one-dimensional continuous fibrous yarns and core-sheath yarns with a modified collector geometry. The electrospun yarns fabricated by this method may be constituted as nano or micro fibers and are suitable for encapsulating various kinds of drugs with high loadings as well as for providing sustained drug release without compromising the mechanical strength (Padmakumar S et al, ACS Appl. Mater. Interfaces, 2016). Such a fibrous system can be utilized to fabricate suturable fibrous implants as implantable drug depots. The present invention utilizes this technology to develop drug laden medical implants by integrating electrospinning with textile technology. This invention combines the advantages of nano/micro fibrous structures and drug loading capability of electrospun fibrous yarns with the tunability in architecture of woven fabrics. The unique architecture of the yarns with numerous internal high surface area interfaces could help in modulating the release profiles of any biological molecule entrapped within the polymeric matrices. This helps to control the combinatory effect of diffusion and matrix degradation which governs the release profiles and thereby enable a sustained release

Peritoneum, being the largest fluid-filled cavity in humans, it is imperative that the drug depot be fixed to the peritoneal wall considering the large number of intra-abdominal and peritoneal organs. Sufficient concentrations of the drug have to be retained in the peritoneal organs and tissues with homogenous distribution of drug throughout the peritoneal cavity. This intraperitoneal drug depot could help to prevent or treat any disease or malignancy that spreads throughout the peritoneal cavity like the cancer of peritoneum, ovary, colon, intestine, pancreas etc. The current clinical strategy of intraperitoneal drug administration is the frequent dosing of drugs into the cavity with indwelling catheters which causes infection and toxicity issues. Hence, an easily implantable intraperitoneal drug delivery system, eliminating the use of catheter would be favorable to both patients and clinicians so as to exploit the pharmacokinetic advantages of intraperitoneal therapy for treating peritoneal malignancies.

The US patent publication US20150080847A1 describes a flexible implantable drug loaded reservoir device for the intraperitoneal treatment of ovarian cancer. The implant is a tubular and elongated device acting as a drug reservoir in solid or semi-solid form which solubilizes the drug in the peritoneal fluid and releases an effective amount continuously for a period of at least 24 hours. These PLLA-based microdevices were seen to be free-floating and migrated towards the pelvic region over the course of treatment in animals (Ye et al., Sustained, low-dose intraperitoneal cisplatin improves treatment outcome in ovarian cancer mouse models. J Control Release. 2015; 220:358-367). Though numerous studies have attempted the development of intraperitoneal drug delivery strategies, there has not been any marketed products or clinically applicable devices with long term effectiveness.

### SUMMARY OF THE INVENTION

The present invention discloses implantable drug delivery devices including suturable fabrics woven from electrospun nano/micro fibrous yarns for providing continuous and sustained release of a drug or biological molecule in a body cavity, method of use thereof and a method of manufacturing thereof.

In one aspect, the invention relates to a woven fabric (100) (also termed a device) of variable packing density, dimension and weight woven from electrospun and continuous fibrous yarn with individual micro- or nano-fibers (105). The fabric is flexible. The yarn includes a single polymer matrix (107) loaded with at least one therapeutic agent (109) at a predetermined concentration. The device is configured to be sutured to the wall of a body cavity of a subject to provide controlled and sustained release of the therapeutic agent.

In some embodiments, the diameter of the electrospun fibrous yarn is in the range of 1 μm to 500 μm. In some embodiments, individual fiber diameter of the electrospun fibrous yarn is in the range of 200 to 2000 nm. In some embodiments, weave pattern of the woven fabric is selected from the group of plain weave, twill weave, satin weave, dobby weave, basket weave, jacquard weave, rib weave, leno weave, oxford weave, or a combination thereof.

In some embodiments, the single polymer is selected from the group of biodegradable polyesters, polyethers, polyanhydrides, polycarbonates, polyphosphazenes, poly(amino acids), polypeptides, glycosaminoglycans, polysaccharides, polydioxanone (PDS), poly(lactide-coglycolide) (PLGA), polyglycolic acid (PGA), polylactic acid (PLA), polycaprolactone (PCL); or non-biodegradable poly(ether urethanes), polystyrene, polyethylene, polyacetylene, poly(propylene), poly(tetrafluroethylene), poly(methymethacrylate), poly(ethylene-co-vinylacetate), poly(dimethylsiloxane), poly(ethylene teraphthalate) and poly(sulphone).

In some embodiments, the at least one therapeutic agent is selected from the group of therapeutic molecules, proteins, paclitaxel, docetaxel, cisplatin, carboplatin, anti-neoplastics, anti-inflammatory, antibiotics, anti-microbial, antiviral agents, analgesics, anti-diabetic agents, antiepileptic agents, anti-estrogens, anti-histamines, anti-parasitic agents, anti-psychotics, anti-pyretics, hormones, peptides, insulin, anti-diuretics, vasodilators, cardio-vascular drugs, immunosuppressive, muscle relaxants, dermatological agents, hematopoietic agents, laxatives, neuromuscular blocking agents, pancreatic enzymes, vitamins, growth factors, signal transduction pathway inhibitors, biological molecule, or a combination thereof. In some embodiments, at least one therapeutic agent is molecularly dispersed in the matrix.

In some embodiments, the device provides controlled and sustained release of the therapeutic agent for duration of at least one month. In some embodiments, the device is configured to be sutured in peritoneal cavity of the subject. In some embodiments, the device is configured to be sutured at multiple locations within the body cavity.

This and other aspects are disclosed herein

### BRIEF DESCRIPTION OF THE DRAWINGS

The invention has other advantages and features which will be more readily apparent from the following detailed description of the invention and the appended claims, when taken in conjunction with the accompanying drawings, in which:

FIGS. 1A, 1B and 1C illustrate a woven fabric implant obtained from electrospun fibrous yarn of single polymer loaded with a therapeutic agent.

FIGS. 2A, 2B, 2C and 2D show SEM images of electrospun polymeric yarns.

FIGS. 3A, 3B and 3C depict photographs of various woven fabrics.

FIGS. 3D, 3E, 3F and 3G show SEM image of various woven fabrics.

FIGS. 4A and 4B show FTIR spectra showing drug encapsulation within polymeric matrices.

FIGS. 5A, 5B and 5C compare the mechanical profile of polymeric yarn with or without paclitaxel drug.

FIGS. 6A, 6B and 6C show the TG/DTA spectra of paclitaxel, polymeric yarn and paclitaxel-loaded polymeric yarn, respectively, demonstrating paclitaxel incorporation does not affect thermal stability of the matrix.

FIG. 7 shows XRD spectra confirming the molecularly dispersed encapsulation of paclitaxel within the semi crystalline polymeric matrix.

FIGS. 8A, 8B, 8C, 8D, 8E and 8F show the SEM images of yarns loaded with paclitaxel that have degraded over a period of 3-4 months after immersion in PBS at pH 7.4, 37° C., and 200 rpm.

FIGS. 9 A, 9B, 9C, 9D, 9E and 9F show the SEM images of the yarns without paclitaxel that have degraded over a period of 5-6 months after immersion in PBS at pH 7.4, 37° C., and 200 rpm.

FIG. 10A shows the in vitro drug release profile of yarns loaded with 2.5, 5, 10 w/w % paclitaxel plotted over a period of 3 months after immersion in PBS at pH 7.4, 37° C., 200 rpm.

FIG. 10B compares the in vitro paclitaxel release profiles of the yarn, 2/2 TP, 1/1 TP and 1/1 LP woven fabrics immersed in PBS at pH 7.4, 37° C. and 200 rpm for about 3 months.

FIG. 10C compares the paclitaxel release profiles of the yarn, 1/1 TP woven fabric and 2/2 TP immersed in peritoneal drain fluid samples derived from ovarian cancer patients, at pH 7.5, 37° C. and 200 rpm.

FIG. 10D compares the paclitaxel release profiles of 1/1 LP and 10 w/w % Paclitaxel PDS yarn immersed in peritoneal drain fluid samples derived from ovarian cancer patients, at pH 7.5, 37° C. and 200 rpm.

FIG. 11A shows the chemical formula of Paclitaxel (PTX) drug of the woven fabric indicating the presence of nitrogen.

FIG. 11B shows EDAX image of the woven fabric.

FIG. 11C EDAX analysis of the woven fabric demonstrating the increase in nitrogen content with increase in drug loading into PDS yarns.

FIG. 12 shows the DSC spectra of Paclitaxel (PTX)-PDS yarns at 0, 30, 90 and 120 days indicating the shift in melting endotherms of degraded PTX-PDS yarns from 105.03° C. to lower temperatures (102.37, 98.19, 93.32° C.) over 30, 90 and 120 days in the course of degradation.

FIGS. 13A, 13B, 13C, 13D, 13E, 13F, 13G, and 13H show images and results from in vivo implantation of woven fabric in mice.

FIGS. 14A, 14B, 14C and 14D shows drug levels in mice implanted with paclitaxel-PDS woven fabric implant.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The term polymeric yarns may refer to electrospun fibrous yarns fabricated from biodegradable polymers.

The term biodegradable fabrics may refer to woven fabrics constituted of fibrous yarns of any biodegradable polymer (surface eroding or bulk eroding) of classes polyesters, polyethers, poly(ether esters, polyanhydrides, polycarbonates, polyphosphazenes; poly(amino acids), polypeptides, glycosaminoglycans and polysaccharides.

The term agent or drug may refer to any therapeutic molecule, drug, protein, biological molecule, or any active pharmaceutical ingredient.

The term intraperitoneal implant may refer to any implantable device which can be implanted within the peritoneal cavity.

The term drug depot refers to any repository of drug which can sustain the drug release profile for several months.

The term ‘suturable’ may refer to any implant that is flexible and that can be sutured into the wall of any body cavity like the peritoneal cavity which elutes the drug from the matrix into the cavity. The implant may be sutured during debulking surgery and the cavity may be closed.

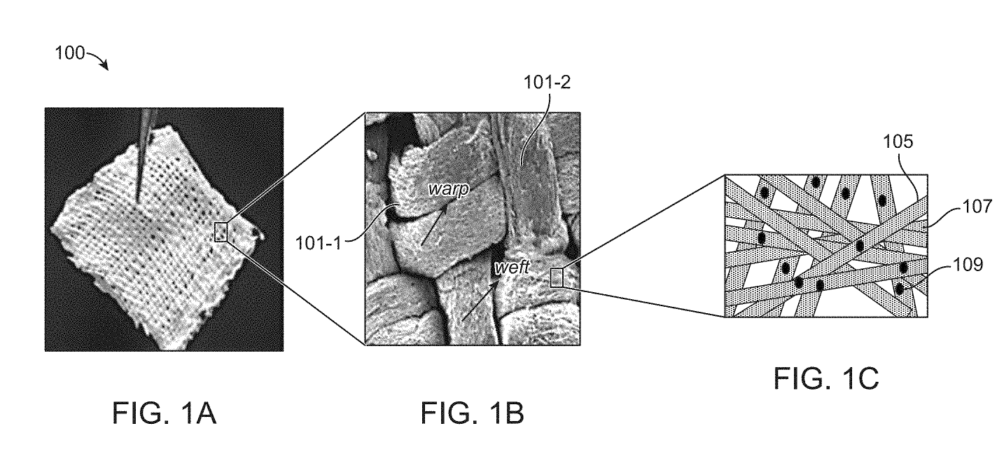

In some embodiments, the present invention relates to a drug delivery device consisting of at least one woven fabric 100 as illustrated in FIG. 1. In some embodiments, the fabric is configured to be suturable in a body cavity. The fabric includes a plurality of warp yarns 101-1 and weft yarns 101-2 obtained from weaving a polymeric yarn. The sutured fabric is configured to be flexible and biodegradable as an implant. In some embodiments, the polymeric yarn is obtained by electrospinning. In some embodiments, the electrospun polymeric yarn includes individual micro- or nano-fibers 105 made of a biodegradable matrix of a single polymer 107 that is loaded with one or more therapeutic agents 109.

In various embodiments, the release of the drug from the woven fabric 100 is controlled and sustained by varying the packing density of the yarn in the fabric. The packing density may be in the range of 10 to 100 mg/cm².

In various embodiments, the length and/or width of the fabric 100 may be in the range of 0.1 cm to 10 m. In some embodiments, the electrospun fibrous yarn 101 may have any suitable suture size known in the art. In some embodiments, suture size is selected from the group of 0, 2-0, 3-0, 4-0, 5-0, 6-0, 7-0, 8-0, 9-0, 10-0, 11-0, or 12-0.

In some embodiments, the fibrous yarns are continuous. In yet another embodiment, the diameter of the electrospun fibrous yarn 101 is in the range of 1 μm to 1000 μm. In yet other embodiments, the diameter is in the range of 1 to 500μm. In some embodiments, length of individual fibers 105 of the electrospun fibrous yarn 101 is in the range of 50 nm to 5000 μm. In other embodiments, the length of individual fiber 105 is between 20 nm to 2 μm.

The fabric 100 may be obtained using any textile technology including weaving method any weaving pattern known in the art. In some embodiments, a conventional weaving loom is used. In some embodiments, a plain weave pattern is used. In other embodiments, twill weave, satin weave, dobby weave, basket weave, jacquard weave, rib weave, leno weave, or oxford weave may be used either separately or in combination with other weaves. In some embodiments, one or more parameters such as the heddle distance and the number of warp yarns to weft yarns maybe varied to obtain the woven fabric 100 with varied packing densities. In some embodiments, the fabric is tightly packed. In other embodiments, the fabric is loosely packed. In some embodiments, the heddle distance is varied in the range of 0.01 to 10 cm to alter the packing density.

In some embodiments, the fibers are in the form of a continuous yarn, a segmented yarn, or a fluffy mass. In some embodiment, the individual fibers are oriented in a random arrangement. In other embodiments, the fibers are aligned in a parallel alignment. In yet other embodiments, the fibers are aligned circumferentially. In some embodiments, the individual fiber is oriented along one plane. In other embodiments, the individual fiber is oriented along multiple planes, including 2 or three planes.

In some embodiments, the mechanical properties such as tensile strength, force and elongation of the polymeric yarns are not affected by the drug loading. In some embodiments, the tensile strength of the fabric is in the range of 5 to 30 MPa. In some embodiments, the tensile strength is at least 3, 6, 9, 12, 15, 30 MPa, or greater. In some embodiments of the disclosure, the thermal stability of the polymeric matrix is not altered by the loading of the therapeutic agent.

In some embodiments, the single polymer 107 is selected from the group of polyesters, polyethers, polyanhydrides, polycarbonates, polyphosphazenes, poly(amino acids), polypeptides, glycosaminoglycans, polysaccharides, polydioxanone (PDS), poly(lactide-coglycolide) (PLGA), polyglycolic acid (PGA), polylactic acid (PLA), or polycaprolactone (PCL). In some embodiments, the single polymer is polydiaoxanone. In some embodiments, non-biodegradable polymers such as poly(ether urethanes), polystyrene, polyethylene, polyacetylene, poly(propylene), poly(tetrafluroethylene), poly(methymethacrylate), poly(ethylene-co-vinyl acetate), poly(dimethylsiloxane), poly(ethylene terphthalate) and poly(sulphone) can be used.

In some embodiments, the at least one therapeutic agent 109 is selected from the group of therapeutic molecules, proteins, paclitaxel, docetaxel, cisplatin, carboplatin, anti-neoplastics, anti-inflammatory, antibiotics, anti-microbial, antiviral agents, analgesics, anti-diabetic agents, antiepileptic agents, anti-estrogens, anti-histamines, anti-parasitic agents, anti-psychotics, anti-pyretics, hormones, peptides like insulin, anti-diuretics, vasodilators, cardio-vascular drugs, immunosuppressive, muscle relaxants, dermatological agents, hematopoietic agents, laxatives, neuromuscular blocking agents, pancreatic enzymes, vitamins, growth factors, signal transduction pathway inhibitors, any biological molecule, or a combination thereof. In one embodiment, the therapeutic agent is a nerve growth factor. In one embodiment, the therapeutic agent is paclitaxel.

The therapeutic agent may be loaded at a concentration typically in the range of 1 to 50 w/w %, more typically in the range of 1 to 20 w/w %. In some embodiments, the at least one therapeutic agent is dispersed molecularly throughout the polymeric matrix upon encapsulation. In some embodiments, the drug encapsulation or loading achieved is at least 80%. In other embodiments, the drug encapsulation is in the range of 85 to 100%.

In some embodiments, the device is configured to be sutured to the wall lining a body cavity to provide controlled and sustained release of the therapeutic agent 109. In yet other embodiments, the device is configured to be sutured in a plurality of locations within any body cavity. In other embodiments, the device is sutured to the walls lining a body cavity including any dorsal, ventral cavity lining, for example peritoneal cavity lining, of a subject to provide controlled and sustained release of the therapeutic agent. In another embodiment, the sutured device prevents metastasis or relapse of a malignancy, such as a peritoneal malignancy, in the subject.

In various embodiments, the woven fabric is suitable and effective for treating a subject with any disorder including cancer, inflammatory, autoimmune or any malignancy, or any hyperproliferative disorder, such as disorder of the ovary, peritoneum or any of the organs in the body cavity of the subject. In some embodiments, the device is used to treat or prevent or mitigate or suppress the condition of the said disorders in a subject. In other embodiments, the device is configured to be sutured in a plurality of locations within the body cavity. In some embodiments, the device is used for treating ovarian cancer by checking the spread of tumour cells by direct contact or via exfoliation from the primary tumour and localized seeding within the peritoneum through peritoneal fluid. In various embodiments, the therapeutic agent released from the implant device is homogenously distributed at an adequate dose to provide an effective treatment for blocking disease spread throughout the organs in the cavity. In some embodiments, the cavity is peritoneal cavity and implant is used to treat or prevent peritoneal malignancy affecting organs like small intestine, large intestine, pancreas, stomach, omentum, mesentery and liver, to which metastasis may occur in peritoneal cancers. In some embodiments, the drug delivery device is additionally coated with a co-polymer, plasticizers, blending, surface coating, or the like. In yet other embodiments, the drug delivery implants are flexible and fixated or sutured to the body cavity such as peritoneal region, is specific and reduces organ toxicity associated with non-fixated implants or systemic therapies.

In various embodiments, the drug release profile is tuned by modulating the packing density of the constituent yarns in the woven fabrics. In some embodiments, the device is capable of releasing in vivo 70 to 80% of the therapeutic agent in about 1 month, 80 to 90% of the therapeutic agent in about 2 months, and/or >90% of the therapeutic agent after 2 months. In other embodiments, the device is capable of releasing in vitro 30 to 40% of the therapeutic agent in about 1 month, 45 to 55% of the therapeutic agent in about 2 months, and/or 55 to 65% of the therapeutic agent in about 3 months. In some embodiments, an initial burse release of about 15% in week 1 is followed by controlled and sustained release for greater than 8 weeks.

In various embodiments, a method of treating a peritoneal disorder or malignancy including cancer, inflammatory, autoimmune or any hyperproliferative disorder of the ovary, peritoneum or any of the organs in the abdominal cavity of the subject by implanting the drug-laden device 100 is provided. The method includes suturing one or more drug-laden devices in the peritoneal wall or lining or peritoneal cavity of the subject in need thereof following cytoreductive surgery. In some embodiments, the sutured device is not associated with infection, migration of implants, inflammation, or fibrous encapsulation of implant.

In various embodiments, a method of fabricating a woven fabric is provided. The method includes obtaining drug-laden yarns by electrospinning a single polymer dissolved in a solution containing at least one organic solvent. The electrospinning solution may additional contain 1 to 50 w/w % of a drug. The conditions for electrospinning may be varied. In one embodiment, the optimized conditions of flow-rate of 2 ml/h, voltage of 12 kV, collector rotation of 700 rpm and yarn-uptake rate of 0.5 m/min is used. In other embodiments, one or more of the flow rate, voltage, collector rotation, and yarn uptake rate are varied. In some embodiments, the encapsulation rate of the drug is 85 to 95%. In some embodiments, the drug is an amorphous drug. In an exemplary embodiment, 8-15 w/v % PDS solution is dissolved in 2, 2, 2-Trifluoroethanol (TFE). In another exemplary embodiment, paclitaxel is loaded at 2.5, 5, and 10 wt % (relative to PDS weight) in a 15 w/v % PDS solution. In one embodiment, the drug-laden electrospun polymeric yarns is fed into a weaving loom for plain weaving by interlacing two sets of drug-loaded yarns as warps and wefts perpendicular to each other on a computer-controlled table top plain weaving loom. In some embodiments, a constant distance was maintained between heddles of the loom to yield loosely packed (LP) woven fabrics. On the other hand, tightly packed (TP) fabrics were woven with no gap between heddles. In some embodiments, the number of warp yarns, weft yarns and the distance between heddles of the loom are altered to fabricate woven fabrics of varied packing densities. In some embodiments, the woven fabrics are cut off from the loom and cut into fabrics of any suitable dimensions (length×width) in the range of 1×1, 2×2, 3×3 cm², etc upto and more than 1×1 m², any dimension there between and of any suitable weight.

In some embodiments, a second polymer sheath solution is electrospun over the prefabricated core yarn wound over a bobbin fitted with a collector. In some embodiments, the second polymer is selected from the group of polyesters, polyethers, polyanhydrides, polycarbonates, polyphosphazenes, poly(amino acids), polypeptides, glycosaminoglycans, polysaccharides, polydioxanone (PDS), poly(lactide-coglycolide) (PLGA), polyglycolic acid (PGA), polylactic acid (PLA), or polycaprolactone (PCL). In some embodiments, the second polymer is PLGA. In some embodiments, the core-sheath polymer reduces or prevents the burst release of the therapeutic agent from the implant.

Without being bound to a particular theory, it is suggested herein that the packing densities of the woven fabrics fabricated using a simple method and by using a single electrospun polymer aid in tuning the polymeric-degradation controlled drug release kinetics of the biodegradable polymers in the body cavity for at least 2 months, 3 months, or greater. The woven fabric based drug delivery devices disclosed herein integrate electrospinning with textile technology. In comparison to techniques with two or more components and complex steps such as blending, coating and co-spinning, a single polymer solution based fibrous yarns obtained sustained release for an extended period through the unique yarn architecture. The advantages of the fabric include its flexibility, biodegradability, nano- or micro-fibrous structures, the high drug encapsulation capability of electrospun fibrous yarns with the tunability in architecture of the woven fabrics resulting in controlled and sustained release of the therapeutic agents in the body cavity for an extended period.

Although the detailed description contains many specifics, these should not be construed as limiting the scope of the invention but merely as illustrating different examples and aspects of the invention. It should be appreciated that the scope of the invention includes other embodiments not discussed herein. Various other modifications, changes and variations which will be apparent to those skilled in the art may be made in the arrangement, operation and details of the system and method of the present invention disclosed herein without departing from the spirit and scope of the invention as described here. While the invention has been disclosed with reference to certain embodiments, it will be understood by those skilled in the art that various changes may be made and equivalents may be substituted without departing from the scope of the invention. In addition, many modifications may be made to adapt to a particular situation or material the teachings of the invention without departing from its scope.

### EXAMPLES

### Example 1: Fabrication of Woven Fabric from Drug-Laden 1D Electrospun Single Polymeric Yarns

A drug-laden medical fabric was developed for implantation by feeding paclitaxel-loaded, one-dimensional uniform and continuous electrospun polymeric yarns with continuous fibers into a weaving loom and a conventional method of plain weaving was adopted for weaving. Woven fabrics were fabricated by interlacing two sets of drug-loaded yarns as warps and wefts perpendicular to each other on a computer-controlled table top plain weaving loom.

The drug-laden yarns were obtained by electrospinning a polydioxanone (PDS) containing organic solution with 1 to 50 w/w % of drug (paclitaxel) at optimized conditions of flow-rate in the range of 1 to 5 ml/h, voltage 10 to 15 kV, collector rotation 500 to 1000 rpm and yarn-uptake rate of 0.25 to 0.75 m/min to obtain 1D continuous yarn with high encapsulation rate of 85 to 95% loaded with amorphous drug within the polymeric matrix. Similarly, other biodegradable single polymer including polyesters, polyethers, polyanhydrides, polycarbonates, polyphosphazenes, poly(amino acids), polypeptides, glycosaminoglycans, polysaccharides, polydioxanone (PDS), poly(lactide-coglycolide) (PLGA), polyglycolic acid (PGA), polylactic acid (PLA), and polycaprolactone (PCL) were prepared and characterized. In one example, 8-15 w/v % PDS solution was dissolved in 2, 2, 2-Trifluoroethanol (TFE). In another example, paclitaxel was loaded at 2.5, 5, and 10 wt. % (relative to PDS weight) in a 10 to 20 w/v % PDS solution.

The single polymeric yarn was smooth, continuous, uniform, mechanically strong and amenable to weaving. The polymeric yarns were fed into a weaving loom. A distance of 0.2 cm was maintained between heddles of the loom to yield loosely packed (LP) woven fabrics. On the other hand, tightly packed (TP) fabrics were woven with no gap between heddles. The number of warp yarns, weft yarns and the distance between heddles of the loom were altered to fabricate woven fabrics of varied packing densities. The woven fabrics were cut off from the loom and cut into fabrics of any dimensions such as 1×1, 2×2, 3×3 cm² and of any weight. The woven fabrics obtained were 1×1 to 10×10 cm² in dimension and 1 mg-100 g in weight. Similarly, any drug may be incorporated in the electrospun yarn.

Core-sheath yarns were fabricated using a similar setup, by using a second polymer sheath (PLGA; 10 w/v %) dissolved in a solvent. The sheath was electrospun over the prefabricated PTX-PDS core yarn wound over a bobbin fitted with a collector.

### Example 2: SEM Characterization of the Drug-Laden Woven Fabric

The fabrics from Example 1 was scanned with Scanning Electron Microscope (SEM) and the average diameters of the electrospun polymeric yarns were measured with and without the drug loaded onto them.

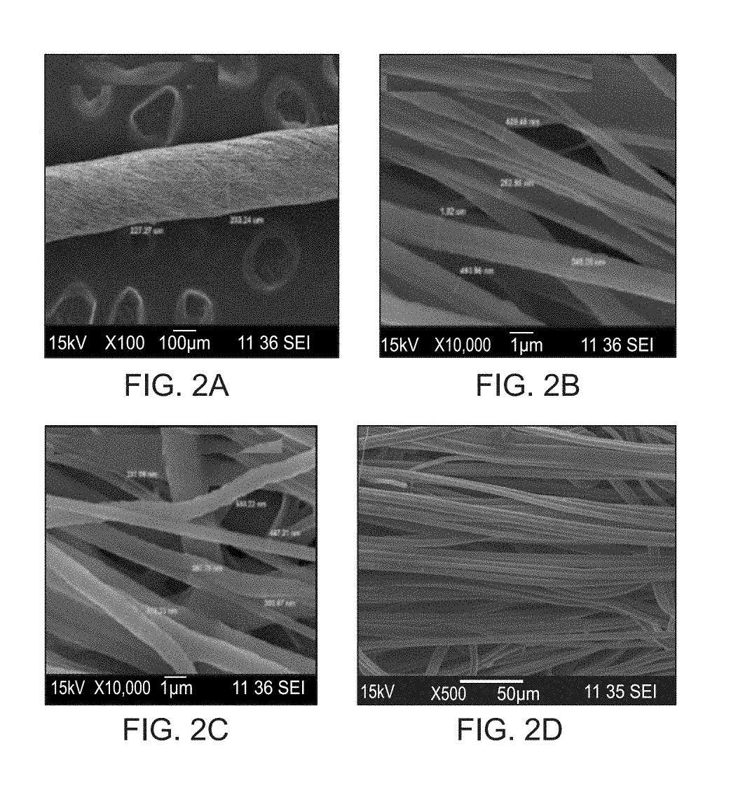

FIGS. 2A-2D show SEM images of electrospun polymeric yarns. FIG. 2A shows the SEM images of electrospun PDS polymeric yarns. It shows smooth, continuous, nano fibrous yarns with an average diameter of 250-280 μm (3-0 sutures USP dimension). The electrospun polymeric yarns with individual fibers having an average diameter of 450 (400-500 nm range) nm are shown in FIG. 2B. FIG. 2C shows the SEM image of 10 w/w % PTX-loaded PDS polymeric yarn having an average diameter of 400-500 nm. FIG. 2D shows SEM image of PTX-loaded PLLA polymeric yarns. PTX inclusion within the PDS yarns did not alter its morphology. Similarly, non-woven electrospun mats with average fiber diameter of ˜750 nm were fabricated by conventional electrospinning for comparison.

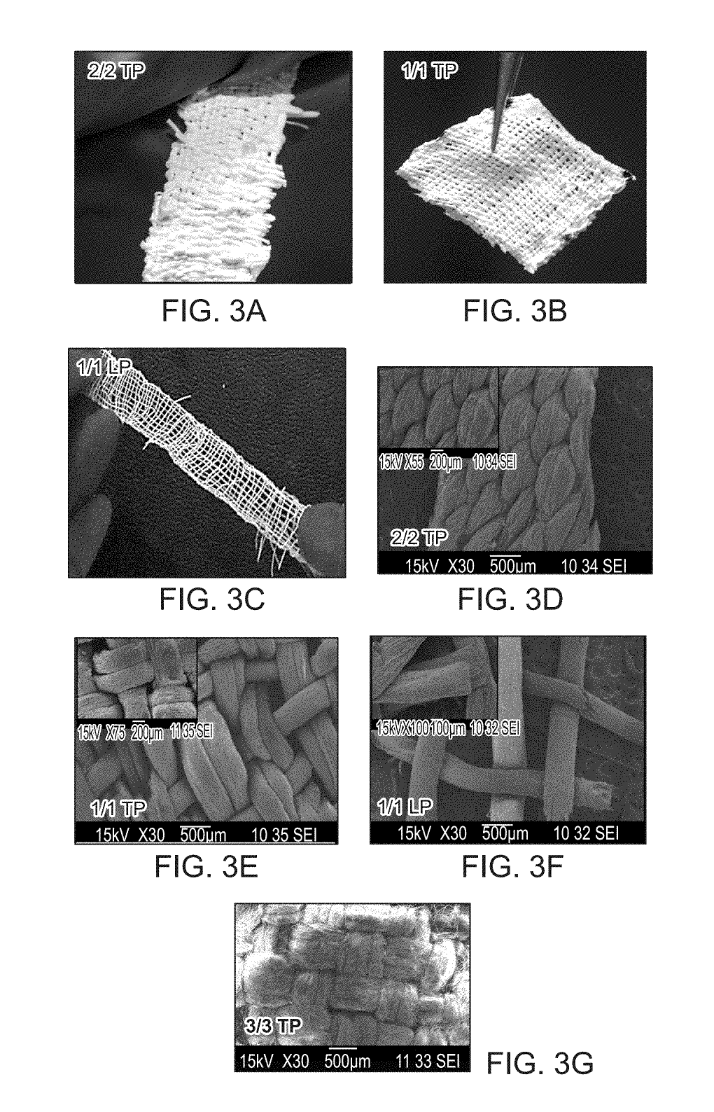

FIGS. 3A-3G show the images of electrospun yarns wound over a bobbin and various fabrics woven using these yarns. The drug laden woven fabric of varying packing densities viz., 1/1 TP (B, E), 2/2 TP (A,D), 1/1 LP (C,F), 3/3 TP (G) (number of warp yarns/weft yarns; TP—tightly packed; LP—loosely packed) were fabricated by loading either one (1/1) or two (2/2) yarns per dent as warp and weft and by changing the number of warp/weft yarns and the heddle distance of the loom. The packing density was in the range of 10 to about 1000 mg/cm². The optical micrographs (A-C) and SEM images (D-G) for the PDS and PLLA based yarns loaded with PTX are shown.

### Example 3: FTIR Testing for Encapsulation of the Drug within the Yarn

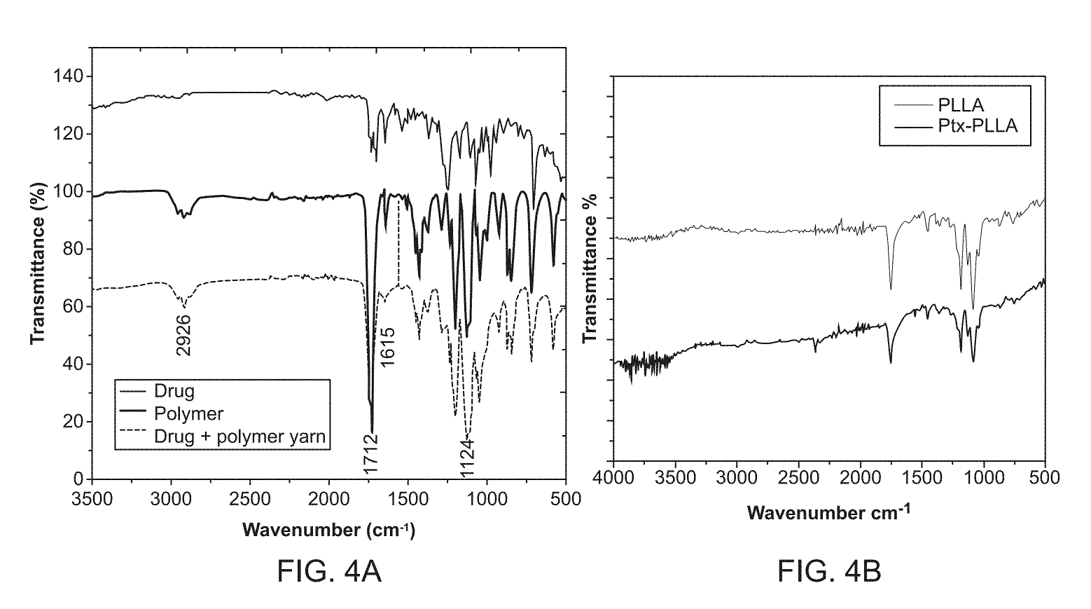

The encapsulation of drug (paclitaxel) within the polymeric matrix of the fabrics from Example 1 was tested using Fourier-Transform Infrared spectroscopy (FTIR) technique. FIG. 4 shows the FTIR spectra of the composite yarns revealing the encapsulation of paclitaxel within the polymeric matrix of PDS (A) and PLLA (B). The retention of CN stretching peak of the drug at 1251 cm⁻¹ was measured to confirm drug entrapment in the polymeric yarns. The peaks at 1128 cm⁻¹ and 1732 cm⁻¹ indicated the ether backbone of PDS polymer and carbonyl groups of both PTX and PDS. The presence of ether-ester backbone of PDS confers greater flexibility as well as elongation to polymeric matrix.

### Example 4: Mechanical Properties Testing of Paclitaxel Loaded Polymeric Yarns

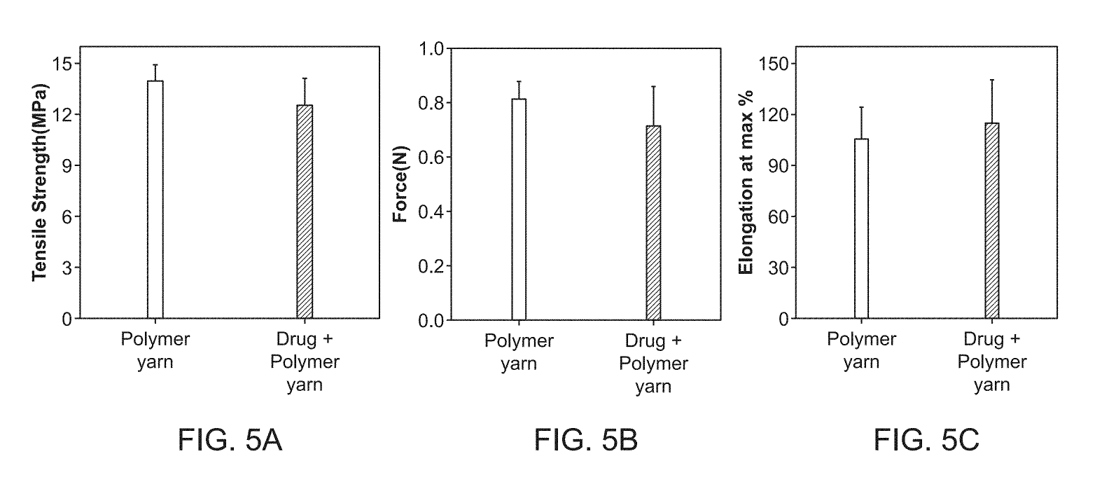

The polymeric yarn without drug and drug-loaded polymeric yarns from Example 1 were tested for their mechanical properties. FIG. 5 shows the mechanical profile of two such yarns. FIG. 5A shows the tensile strength testing, FIG. 5B shows the force testing and FIG. 5C shows the elongation testing of the polymeric yarns. As shown in the images, loading of drug did not alter the mechanical properties such as tensile strength, force and elongation of the yarns drastically. PTX-PDS yarns with a force at break of ˜0.8N were amenable to plain weaving for developing implantable textiles.

### Example 5: XRD Spectra of Paclitaxel, Polymeric Yarn and Paclitaxel Loaded Polymeric Yarn

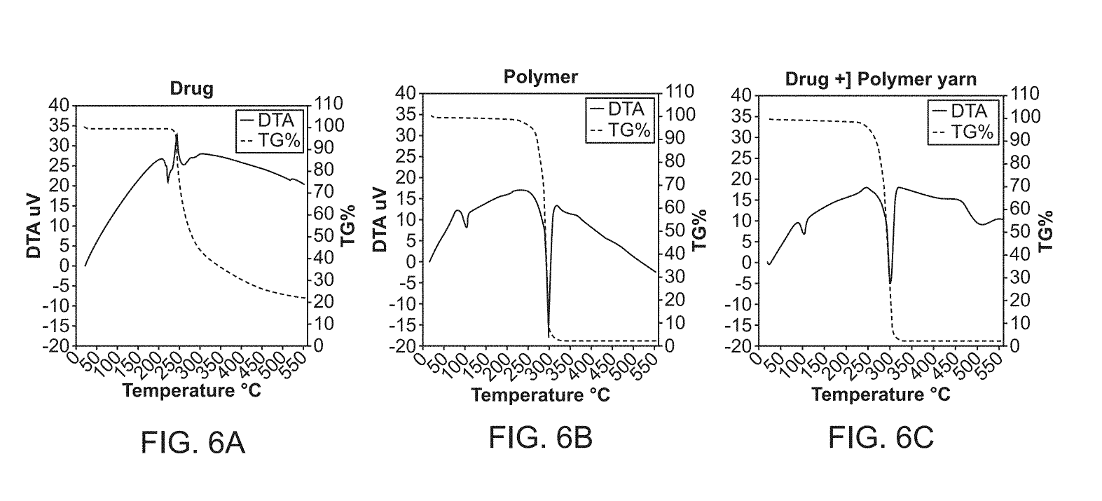

FIGS. 6A-6C show the XRD spectra of paclitaxel (Drug), polymer and paclitaxel loaded polymeric yarns, respectively. The semi-crystalline nature of the polymer is shown with characteristic sharp peaks at 22.7° and 24.38°. Broad peaks in the spectrum of drug loaded yarns confirm the amorphous encapsulation of drug in the polymeric yarns.

### Example 6: TG/DTA Thermograms of Paclitaxel, Polymeric Yarn and Paclitaxel Loaded Polymeric Yarn

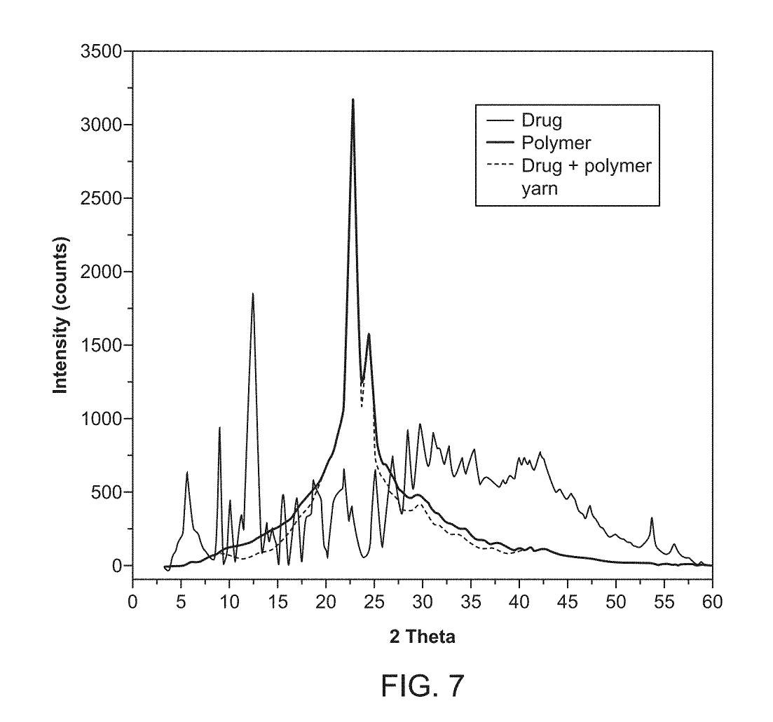

The paclitaxel (Drug), polymeric yarn and the paclitaxel loaded polymeric yarn were tested for their thermal stability. FIG. 7 shows the TG/DTA thermograms. From the thermograms, it can be inferred that the thermal stability of the polymeric matrix was not altered by drug loading.

### Example 7: Drug Release Profile Testing

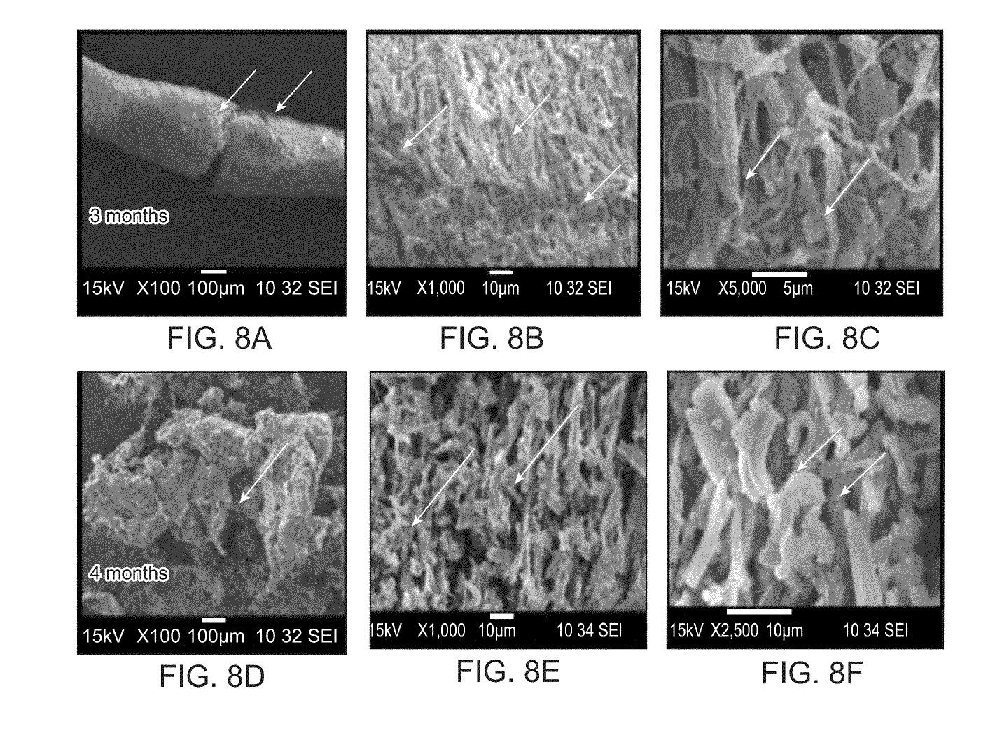

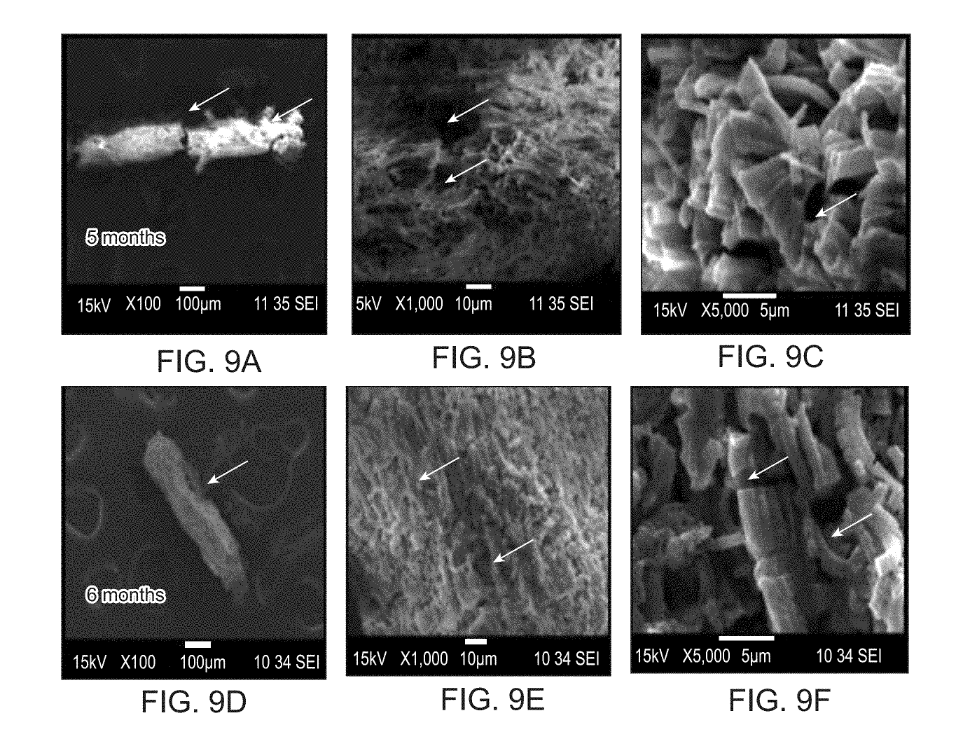

The matrix degradation was observed over a period of 4 to 6 months for the fabrics of Example 1. FIGS. 8A-8F show the SEM images of paclitaxel loaded polymeric yarns immersed in PBS at pH 7.4, 37° C., 200 rpm. FIGS. 9A-9F show the SEM images of polymeric yarns without paclitaxel immersed in PBS at pH 7.4, 37° C., 200 rpm. It was observed that the in vitro degradation rate was accelerated by the presence of the drug, denoted by the fragmentation of fibers and the complete rupture of the drug loaded yarns by 4 months. PTX-PDS yarns completed degradation within 120 days into much smaller fragments than that of PDS yarns at 120 days.

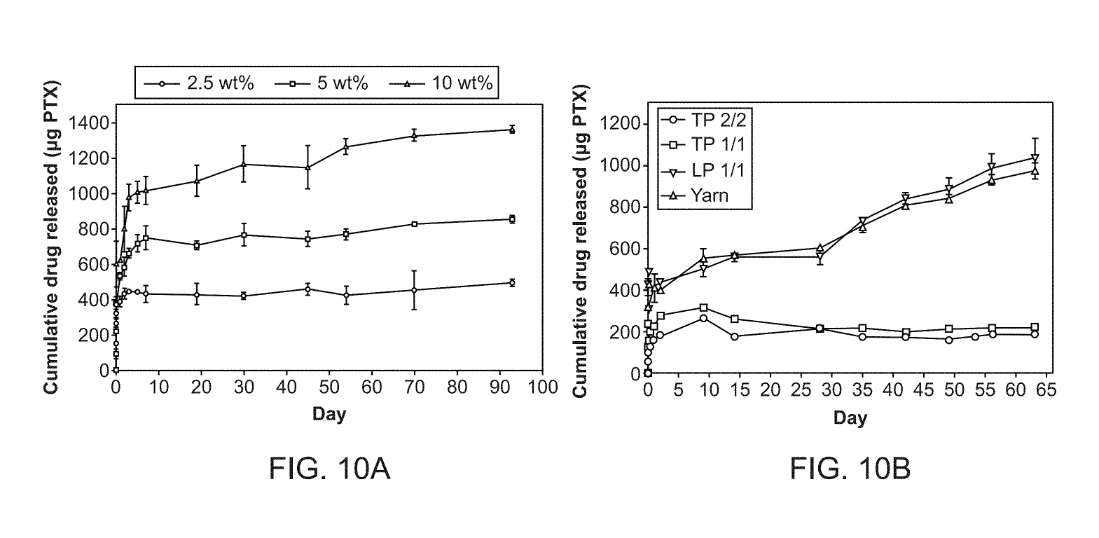

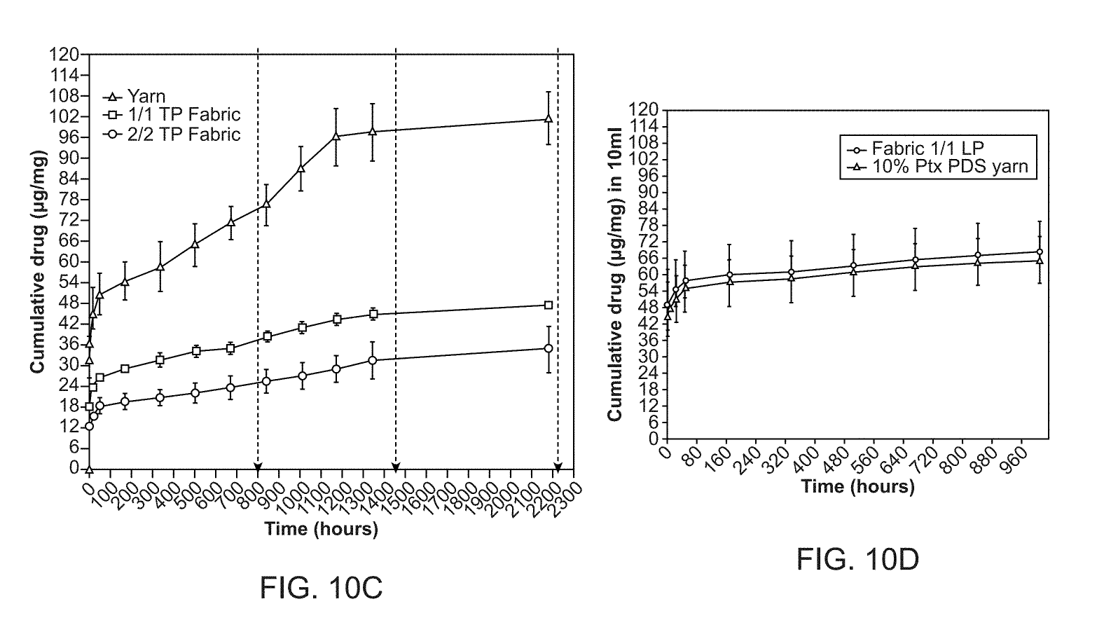

FIG. 10A shows the in vitro drug release profiles from polymeric yarns loaded with 2.5, 5, 10 w/w % of paclitaxel in PBS—pH 7.4, 37° C., 200 rpm. Higher the drug loading, higher is the drug release. All the yarn samples showed a sustained drug release profile for up to three months. This data correlates with the degradation profile of drug loaded yarns.

FIG. 10B shows the in vitro cumulative drug release profiles of polymeric yarns loaded with 10 w/w % paclitaxel and 2/2 TP, 1/1 TP and 1/1 LP fabrics woven with these yarns in PBS—pH 7.4, 37° C., 200 rpm. The drug release profiles are sustained for almost 3 months. 2/2 TP fabrics show the slowest drug release profile due to the high packing density of yarns in the fabric providing less surface exposure of the fibers to the release media, for enabling hydrolysis. Moderate release profile is shown by 1/1 TP fabric due to the relatively lesser packing density, thereby providing more surface exposure for the fibers. 1/1 LP shows release similar to that of yarns as the woven fabric is loosely packed.

FIGS. 10C and 10D show the drug release profiles from single polymeric yarns loaded with 10 w/w % paclitaxel weighing 20 mg as well as 20 mg weighing fabrics (1/1, 2/2 TP and 1/1 LP fabrics) woven with these yarns, in patient derived peritoneal drain fluid samples collected post cyto-reductive surgery. 2/2 TP fabrics shows the slowest drug release profile similar to that of in vitro drug release profiles in PBS. 1/1 LP fabrics shows drug release profiles similar to that of drug loaded yarns.

### Example 8: EDAX Analysis of Yarns Loaded with Different Drug Loadings

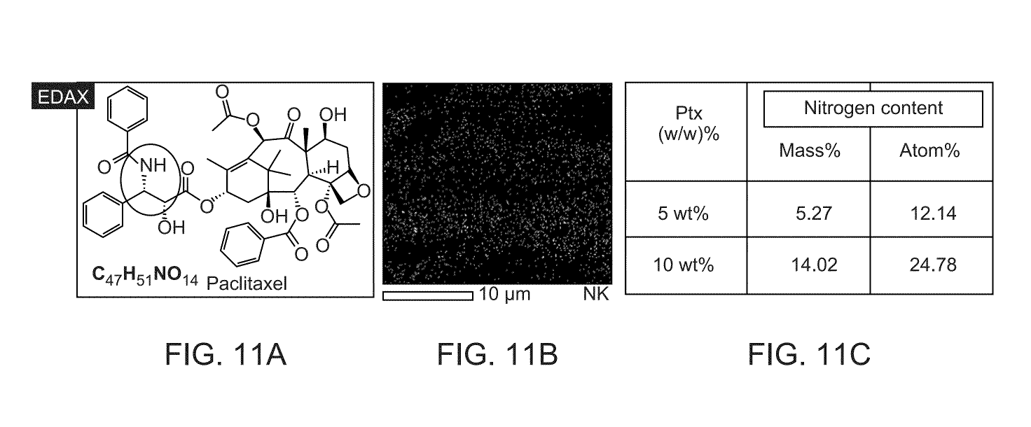

FIG. 11 shows the chemical formula of Paclitaxel (PTX) drug indicating the presence of nitrogen, EDAX analysis demonstrating the increase in nitrogen content with increase in drug loading into PDS yarns confirming the qualitative encapsulation of paclitaxel in PDS yarns. Quantitatively, PTX entrapment efficiency in PDS yarns was estimated by HPLC to be 86±10%.

### Example 9: Thermal Analysis of Degraded Yarns

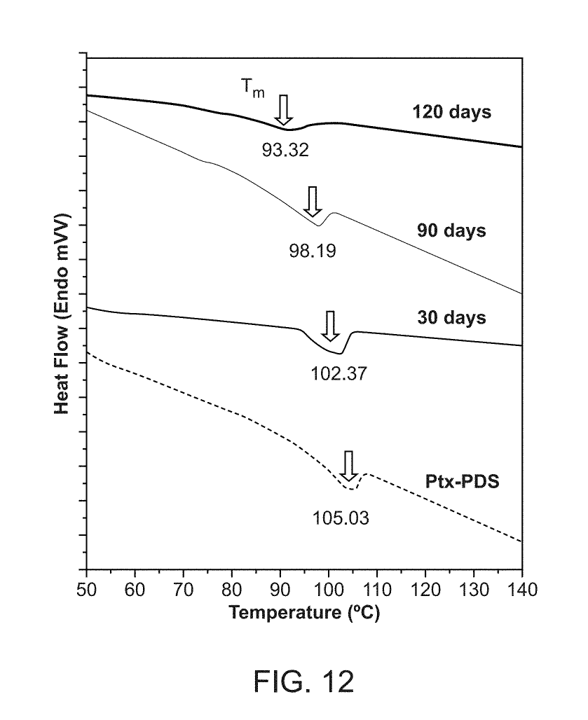

FIG. 12 shows the DSC spectra of Paclitaxel (PTX)-PDS yarns at 0, 30, 90 and 120 days indicating the shift in melting endotherms of degraded PTX-PDS yarns from 105.03° C. to lower temperatures (102.37, 98.19, 93.32° C.) over 30, 90 and 120 days in the course of degradation. It is known that, during hydrolytic degradation, water entry into the amorphous regions of PDS would be faster than its crystalline regions. This would lead to chain scission of hydrolytically unstable ester bonds found in amorphous parts of PDS which ultimately reduces the molecular weight of polymer, which is reflected in the shift in melting endotherms of degraded yarns.

### Example 10: In Vivo Implantation of Woven Fabric in Mice

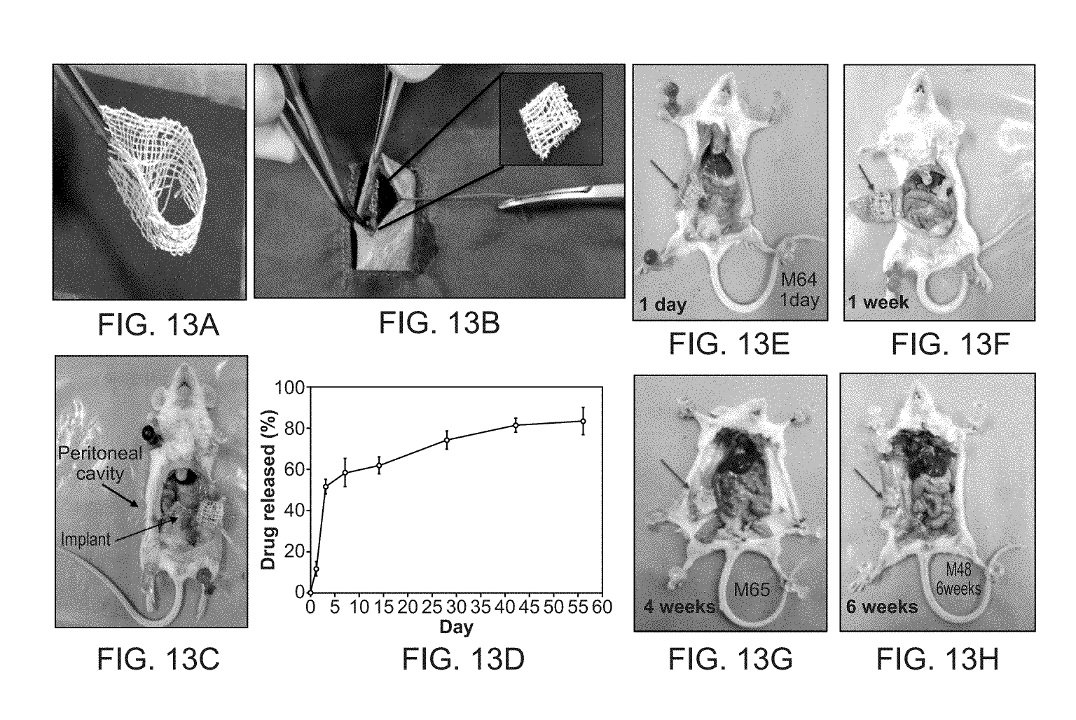

FIGS. 13A-13C show the in vivo implantation of woven fabric in mice. FIG. 13A shows a photograph of flexible lace of loosely packed woven fabric after plain weaving process. FIG. 13B shows surgical implantation of fabric into healthy BALB/c mouse (inset: 1×1 cm² 1/1 LP fabric implant weighing 20 mg) by suturing to mouse peritoneal cavity wall and subsequent closure of the peritoneal wall and skin. FIG. 13C shows post mortem view of the mouse peritoneum upon euthanasia at 1 week showing the intact implant fixed at its sutured site.

The implants remained intact at sutured site at all euthanasia time points (1, 3, 7, 14, 28, 42, 56 days). Post-mortem examination of peritoneum revealed no signs of infection, inflammation or fibrous encapsulation of implant. In contrast to woven nanotextile, non-woven electrospun PTX-PDS mats could not be sutured along the peritoneal wall owing to their inferior mechanical integrity. When placed without suturing in peritoneal cavity, mats migrated to distant locations and hence were fixed to peritoneal wall by a surgical suture-knot. However, these animals could not survive for more than 4 weeks, with considerably reduced food intake and fecal excretion. Post-mortem examination of morbid animals revealed the migration and attachment of implant to peritoneal organs, mainly the intestine, with high fibrous encapsulation of implant, bowel obstruction, swollen-abdomen with blood-filled cavity and feces accumulation. Similarly, the exemplified implant may be implanted in any body cavity.

### Example 11: In Vivo Drug Release of Woven Fabric in Mice

FIG. 13D shows the in vivo Paclitaxel release profile until 8 weeks (n=5/time point) analyzed by HPLC showing the sustained drug release profile from woven fabric in mice peritoneal cavity for 2 months with 83.4±6.47% of total drug released in about 2 months and thereby indicating the possibility to elute further.

Although release rates were somewhat higher than for the in vitro case, sustained release over a sixty day period could still be obtained which has not been possible with conventional polymers before. 11.67±3.77% of encapsulated-PTX was released on day 1 and 51.67±3.44% on day 3. This initial burst, which could be compared to PTX loading dose administered in current IP therapy is a benefit, owing to the PK advantages of PTX, giving enhanced therapeutic efficacy. The initial burst was followed by a steady increase in drug concentration with nearly 83.4±6.47% of total PTX content released within 8 weeks, implying its potential to elute drug further.

### Example 12: In Vivo Long Term Stability of Woven Fabric in Mice

FIGS. 13E-13H show the photographs of mice implanted with woven fabric implant (indicated by arrow marks) euthanized at different time points until 8 weeks. The fabric remains intact at the sutured location throughout the treatment period without intense fibrous tissue formation around the implant.

### Example 12: In Vivo Biodistribution of Paclitaxel Woven Fabric in Mice in Comparison to IP Taxol Injected Mice

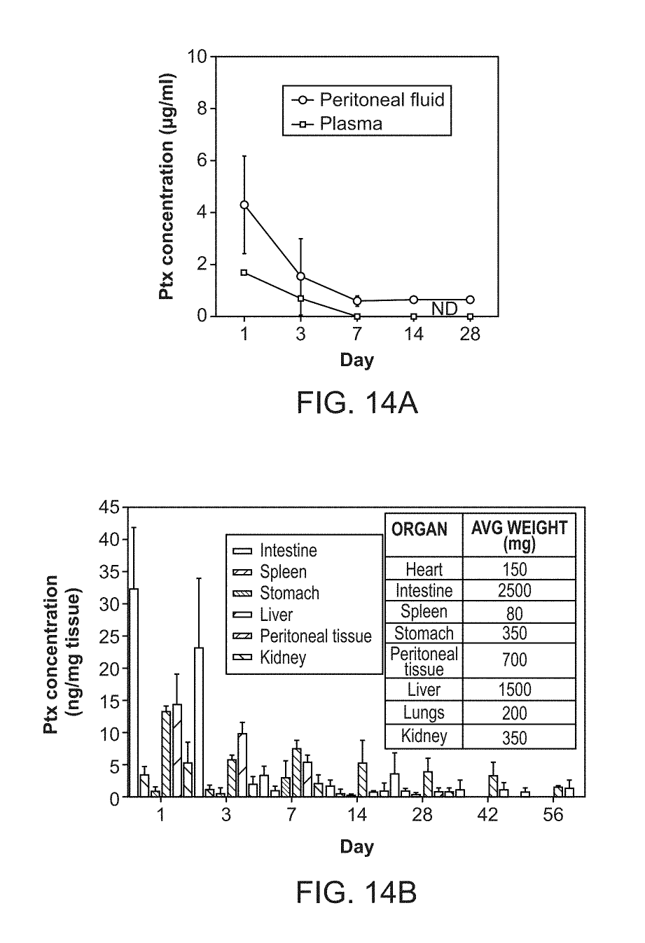

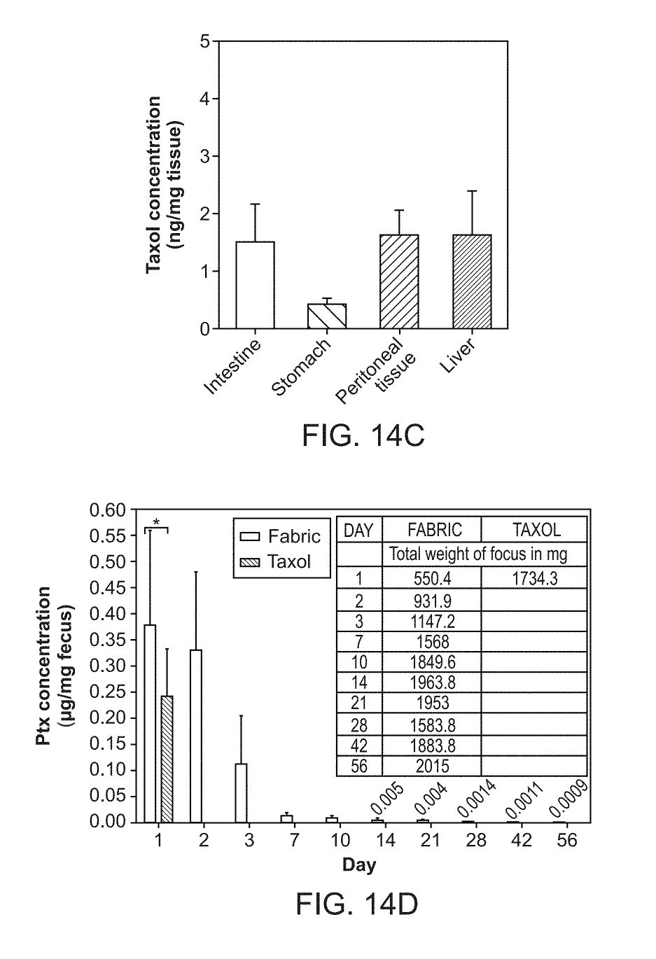

FIGS. 14A-14D show the drug levels in mice implanted with Paclitaxel-PDS woven fabric implant at various time points analyzed by HPLC (A) peritoneal lavage and plasma samples (B) various tissues (inset table: average weight of organs after dissection) (n=5). (C) Drug biodistribution in mice injected with IP Taxol as control group (20 mg/kg) at day 1 (n=5). (D) Drug excretion profile of mice with woven fabric and IP Taxol (inset table: average feces weight at various time points). Data shown as mean±SD, *p<0.05.

Peritoneal lavage collected post-implantation on days 1, 3 and 7 yielded PTX concentrations of 4.31±1.9, 1.54±1.5 and 0.6±0.21 μm/ml respectively, indicating high PTX retention in peritoneum relative to plasma, and therefore the PK advantage of IP therapy. PTX distribution to various vital organs was analyzed post implantation of woven nanotextile up to 8 weeks shows PTX peaks in all peritoneal organs (intestine, spleen, stomach, liver, and kidney) till day 56, with no traces of PTX in heart and lungs. Thus, the extended drug release from woven nanotextile was also evident from PTX biodistribution profile.

Mice injected with IP taxol did not show Paclitaxel in plasma or peritoneal lavage at 24 hours and thereafter, indicating the rapid clearance of the drug. The sustained drug levels in plasma and peritoneal lavage were noted for mice bearing woven fabric. Peritoneal organs showed drug peaks only for day 1 for mice injected with IP Taxol whereas all peritoneal organs retained drug peaks throughout the treatment period (8 weeks) for mice implanted with woven fabric implants. This result was also observed for drug peaks in feces, confirming the sustained drug release from woven fabric when implanted in vivo.

What is claimed is:

1. A device, comprising: a flexible woven fabric (100) having a variable packing density, dimension and weight, the fabric comprising electrospun continuous fibrous yarn with individual micro- or nano-fibers (105), the fiber comprising a single polymer matrix (107) loaded with at least one therapeutic agent (109), wherein the at least one therapeutic agent is loaded in the single polymer matrix at a predetermined concentration, and wherein the device is configured to be sutured to the wall of a body cavity of a subject to provide controlled and sustained release of the therapeutic agent.

2. The device of claim 1, wherein diameter of the electrospun fibrous yarn is in the range of 1 μm to 500 μm and individual fiber diameter of the electrospun fibrous yarn is in the range of 200 nm to 2000 nm.

3. The device of claim 1, wherein weave pattern of the first woven fabric is selected from the group of plain weave, twill weave, satin weave, dobby weave, basket weave, jacquard weave, rib weave, lend weave, oxford weave, or a combination thereof.

4. The device of claim 1, wherein the device provides controlled and sustained release of the therapeutic agent for at least 1 month.

5. The device of claim 1, wherein the single polymer is selected from the group of polyesters, polyethers, polyanhydrides, polycarbonates, polyphosphazenes, poly(amino acids), polypeptides, glycosaminoglycans, polysaccharides, polydioxanone (PDS), poly(lactide-coglycolide) (PLGA), polyglycolic acid (PGA), polylactic acid (PLA), or polycaprolactone (PCL) or the non-biodegradable polymers such as poly(ether urethanes), polystyrene, polyethylene, polyacetylene, poly(propylene), poly(tetrafluroethylene), poly(methymethacrylate), poly(ethylene-co-vinylacetate), poly(dimethylsiloxane), poly(ethylene teraphthalate) and poly(sulphone).

6. The device of claim 1, wherein at least one therapeutic agent is selected from the group of therapeutic molecules, proteins, paclitaxel, docetaxel, cisplatin, carboplatin, anti-neoplastics, anti-inflammatory, antibiotics, anti-microbial, antiviral agents, analgesics, anti-diabetic agents, antiepileptic agents, anti-estrogens, anti-histamines, anti-parasitic agents, anti-psychotics, anti-pyretics, hormones, peptides, insulin, anti-diuretics, vasodilators, cardio-vascular drugs, immunosuppressive, muscle relaxants, dermatological agents, hematopoietic agents, laxatives, neuromuscular blocking agents, pancreatic enzymes, vitamins, growth factors, signal transduction pathway inhibitors, or a combination thereof.

7. The device of claim 1, wherein the device is configured to be sutured to the wall of peritoneal cavity.

8. The device of claim 1, wherein the device is configured to be sutured at multiple locations within the body cavity.
